(19) United States
(12) Patent Application Publication
(10) Pub. No.: US 2009/0298844 A1
(43) Pub. Date: Dec. 3, 2009

(54) ABL KINASE INHIBITION

(76) Inventor: John Pollard, Oxfordshire (GB)

Correspondence Address: VERTEX PHARMACEUTICALS INC., 130 WAVERLY STREET, CAMBRIDGE, MA, 02139-4242, US

(21) Appl. No.: 11/989,300

(22) PCT Filed: Jul. 26, 2006
(86) PCT No.: PCT/US06/28984

§ 371 Date: Jun. 12, 2009

Related U.S. Application Data
(60) Provisional application No. 60/702,771, filed on Jul. 26, 2005.
(60) Provisional application No. 60/785,161, filed on Mar. 23, 2006.
(60) Provisional application No. 60/830,257, filed on Jul. 12, 2006.

(51) Int. Cl. A61K 31/48; C12N 9/99

(52) U.S. Cl. 514/252.19; 435/184

(57) ABSTRACT

The present invention relates to inhibition of AbI kinase.

### FIELD OF THE INVENTION

This invention relates to inhibition of Abl kinase.

### BACKGROUND OF THE INVENTION

Chronic myeloid leukemia (CML) is a malignant disorder of haematopoietic stem cells which affects 1-2 people per 100,000 and constitutes about 15% of all adult leukemias.

Imatinib is the front line therapy for CML, and its primary mechanism of action has been demonstrated to be through inhibition of the tyrosine kinase activity of the Bcr-Abl fusion protein. About 90% of patients with chronic phase CML respond to Imatinib with about 50% of those showing a cytogenic response (normalization of blood counts and loss of the Philadelphia chromosome). The remaining 50% show a hematologic response (normalization of blood counts with retention of the Philadelphia chromosome) but ultimately many relapse with re-growth of hematopoeitic elements associated with primary resistance to the drug (Cancer Cell, 2002, 2, 99: Cancer Cell, 2002, 2, 117).

In the advanced blastic phase of the disease the response to Imatinib is reduced to about 60% of patients, however almost all of these ultimately relapse (Cancer cell 2002, 2, 117). Resistance commonly arises through selection of point mutations in the BCR-Abl protein. Amongst the most common is the T315I mutation which accounts for ˜20% of the mutant population. Consequently there is a great deal of interest across the CML and ALL (acute myeloid leukemia) communities to identify BCR-Abl inhibitors that are able to block the activity of these mutations and in particular the T315I mutant. To date seven Abl inhibitors are progressing through clinical trials. Six of these act by competing with ATP for the substrate binding pocket and whilst they commonly show good activity against wild type Abl and multiple Abl mutations, none of these show any inhibition of the T315I mutant. ONO12380 is a non-ATP competitive inhibitor of Abl kinase activity and represents the only compound known to inhibit the T315I mutant.

Resistance to Imatinib is reported to arise through one of two mechanisms, either overexpression of Bcr-Abl as a result of gene amplification or, more frequently, selection of specific point mutations within the Abl kinase domain (Cancer Cell, 2002, 2, 117). Crystallography studies have shown that Imatinib binds to the ATP pocket of the kinase when the activation loop is in the closed conformation, as such the compound binds and stabilises an inactive kinase conformation (Science, 2000, 289, 1938). To date over 30 point mutations have been identified which confer resistance to Imatinib either by directly disrupting the interaction between the protein and the inhibitor or by stabilising an open kinase conformation. This commonly results in constitutive enzyme activation and a protein conformation that lacks the key lipophilic pocket required for Imatinib binding (Cancer Cell, 2005, 7, 129). One of the most common mutations is a Threonine to Isoleucine change at residue 315 (T315I), which accounts for 15-20% of the Bcr-Abl mutations. Transfection of the T315I mutant in the Il-3 dependent BaF3 cell line promotes growth in the absence of the mitogen and renders the cells resistance to Imatinib (IC50 for viable cell count of >10 μM vs. 0.6 μM in cells transfected with wt Bcr-Abl) (Cancer cell, 2002, 2, 117).

A number of Bcr-Abl inhibitors have been identified and tested in clinical trials of CML (see Table 1). Although these inhibitors typically show increased efficacy against both wt Bcr-Abl and many of the Imatinib resistant mutations, none have yet been reported to have activity against this common T315I mutant (Haematologica, 2005, 90, 534). This data represents a highly significant finding since this specific mutation, commonly observed in Imatinib resistant CML, renders the Abl kinase resistant to all of the known agents progressing through the clinic (Table 1).

TABLE 1
Table 1. Small molecule protein kinase inhibitors currently being
pursued in Imatinib resistant CML (Haematologica, 2005, 90, 534).

| Agent | Company | Target | Clinical stage |
|---|---|---|---|
| SKI-606 | Wyeth-Ayerst | Abl, Src | I |
| BMS354825 | Bristol-Myers | Abl, Src | II |
| (Dasatinib) | | | |
| AZD0530 | Astra Zeneca | Abl, Src | Pre-clinical |
| AP23464 | Ariad | Abl, Src | Pre-clinical |
| CGP76030 | Pfizer | Src | Pre-clinical |
| AMN107 | Novartis | PDGFR, Abl, Kit | I-II |
| (Nilotinib) | | | |

There is therefore a need for compounds that can inhibit Abl kinase, mutant forms of Abl kinase, and the T315I mutant form Abl kinase.

### SUMMARY OF THE INVENTION

This invention relates to inhibition of Abl kinase, including mutant forms of the kinase. In one embodiment, this invention relates to inhibition of Abl kinase having a T315I mutation.

### DETAILED DESCRIPTION OF THE INVENTION

The present invention provides methods for inhibiting an Abl kinase, including wild type Abl kinase and mutant forms of Abl kinase. In certain embodiments, the invention provides methods for inhibiting Abl kinase having a T315I mutation.

Applicants have demonstrated that Compound I (otherwise known as VX-680 or MK-0457) is a potent inhibitor of both wild type Abl kinase activity and the T315I mutant with inhibition constants of 30 and 42 nM respectively. Compound I is a potent small molecule inhibitor of the Aurora family of protein kinases that is currently in phase I clinical trials. Compound I demonstrates excellent selectivity against over 60 other protein kinases tested, with potent cross-reactivity against only Flt-3 (a receptor tyrosine kinase commonly constitutively activated in acute myeloid leukemia (Cell Mol Life Sci, 2004, 61, 2932: Mini Rev Med Chem, 2004, 4, 255). Compound I causes apoptotic cell death in vitro and tumor regression vivo at well-tolerated doses in xenograft animal models of AML and colon cancer (HL-60 and Hct166 respectively) (Nat Med, 2004, 10, 262).

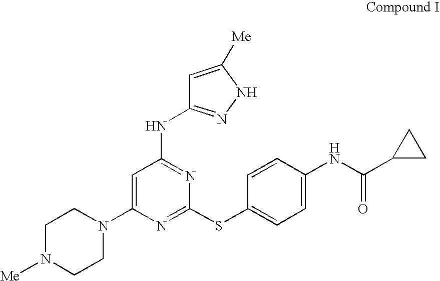

Compound I is highly potent inhibitor of recombinant purified Abl kinase activity with an inhibition constant (Ki) of 30 nM. This compares with Ki for Aurora-A of 0.6 nM, 18 nM for Aurora-B, 4.6 nM for Aurora-C and 30 nM for Flt-3 (Nat Med, 2004, 10, 262). Compound I binds a conformation of Aurora-A that is reminiscent of the conformation of Abl bound to Imatinib.

Accordingly, one embodiment of this invention provides a method for inhibiting Abl kinase, comprising contacting Compound I and the Abl kinase.

In certain embodiments, the Abl kinase is in a patient in need of Abl kinase inhibition and the method comprises administering a therapeutically effective amount of Compound I to the patient.

This invention also provides a method treating a patient having CML, comprising administering to the patient a therapeutically effective amount of Compound I, or a pharmaceutically acceptable salt thereof.

This invention also provides a method treating a patient having ALL, comprising administering to the patient a therapeutically effective amount of Compound I, or a pharmaceutically acceptable salt thereof.

Additionally, Compound I is a highly potent inhibitor of the most common Imatinib resistant Abl mutant (T315I). Highly potent inhibition against the recombinant protein was observed with a measured IC50 of 70 nM corresponding to an estimated Ki of 42 nM (residual enzyme activity was refitted to the Morrison equation for tight binding inhibition using a value of 17 μM for Km), which is comparable to the observed inhibition against wild type Abl.

Compound I has also been shown to be effective in patients in vivo (see Example 7).

Accordingly, in certain embodiments of this invention, the Abl kinase is wild-type kinase. In other embodiments, the Abl kinase is a mutant form of the Abl kinase. In still other embodiments, the mutant form of the Abl kinase is a T315I mutant.

This invention also provides therapeutic methods comprising the steps of determining whether a T315I Abl mutation is present in a patient (particularly, a patient having CML or ALL) and, if the T315I Abl mutation is present, administering Compound I to the patient.

Compound I may be synthesized according to the General Scheme and Examples herein (see also WO 04/000833, which is incorporated herein by reference). Additionally, Compound I may be synthesized by methods known to skilled practitioners.

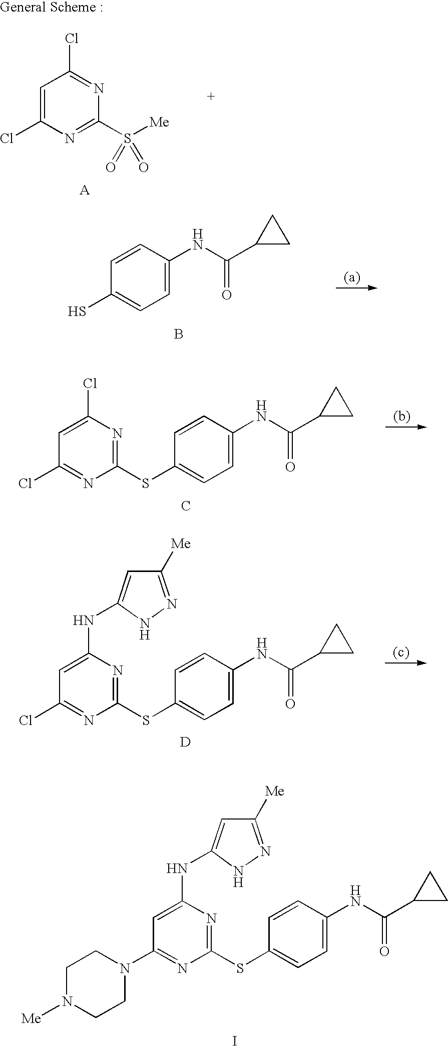

In another embodiment, this invention provides pharmaceutical compositions comprising Compound I and a pharmaceutically acceptable carrier, adjuvant or vehicle.

A “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutically acceptable salts of Compound I include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining Compound I and pharmaceutically acceptable acid addition salts thereof.

Salts derived from appropriate bases include alkali metal (e.g., sodium and potassium), alkaline earth metal (e.g., magnesium), ammonium and N⁺(C₁₋₄ alkyl)₄ salts. This invention also envisions the quaternization of any basic nitrogen-containing groups of Compound I. Water or oil-soluble or dispersible products may be obtained by such quaternization.

For examples of specific salts of Compound I, see WO 04/000833.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of Compound I include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration.

Alternatively, pharmaceutically acceptable compositions of this invention are formulated for IV administration.

The amount of Compound I that may be combined with the carrier materials to produce a composition in a single dosage form will vary depending upon the host treated, the particular mode of administration. Preferably, the compositions should be formulated so that a dosage of between 0.01-100 mg/kg body weight/day of the compound can be administered to a patient receiving these compositions.

A 20 mg/mL lactic acid formulation of Compound I (also known as VX-680 or MK-0457) may be prepared according to the following steps: Prepare a 20 mg/mL concentration of lactic acid in water by weighing 2.0 g of lactic acid (either L-lactic acid, D-lactic acid or a racemic mixture) into a 100 mL volumetric flask. Next, weigh out 200 mg of Compound I into a 10 mL volumetric flask. Next, add approximately 8 mL of the 20 mg/mL lactic acid solution to the 10 mL volumetric flask. Next, add the appropriate amount of sugar (for example, 15 mg/mL, 50 mg/mL or 100 mg/mL, depending on the desired tonicity). Stir the solution until all the drug contents are dissolved. Qs'd the solution to 10 mL with the 20 mg/mL lactic acid solution and adjust the pH as needed to aid in solublization.

A 20 mg/mL lactic acid formulation of Compound I (large scale manufacture) may be prepared according to the following steps: Add water for injection equal to 80 percent of batch weight to a suitable mixing vessel. Add the necessary amount of compendial lactic acid (either L-lactic acid, D-lactic acid or a racemic mixture) equaling to 20 mg/mL and mix to insure homogeneity. Add Compound I equal to 20 mg/mL free base to the vessel and mix to dissolve. Add the appropriate amount of sugar (for example, 15 mg/mL, 50 mg/mL or 100 mg/mL, depending on the desired tonicity) to the vessel and mix to dissolve. Adjust the pH as needed. Qs'd the batch to final weight with water for injection. Sterile filter and collect the filtered formulation in an appropriate sterile receiving vessel. Fill and stopper the formulation in appropriate vials using aseptic technique in a properly classified area. Cap and terminally sterilize product as required. Store the formulation at the appropriate temperature conditions.

A lyophilized powder formulation for reconstitution with sterile water for injection may be prepared according to the following steps: Place approximately 90% of the final batch weight of water for injection, USP into a tared, clean agitated vessel. Add the specified amount of mannitol, USP; agitate for at least 15 minutes to dissolve. Add the specified amount of the sulfate salt of Compound I; agitate for at least 30 minutes to dissolve. Add water for injection, USP to the final batch weight. For purposes of this examplary formulation, the final batch contains the following proportions:

| Component | mg/mL | mg/vial |
|---|---|---|
| Compound I-sulfate | 12.1 | 91.0 |
| (as equivalent free base) | (10.0) | (75.0) |
| Mannitol | 50 | 375 |
| Water for Injection | q.s. to 1.0 mL | q.s. to 7.5 mL |

Cool the solution thus prepared to 22° C. and filter through a 0.22 μm sterilizing filter into appropriate sterile containers. Lyophilize to form a white powder.

The sulfate salt of Compound I (dry powder) may be prepared according to the following steps: To Compound I in solution in ethanol at 70° C. (7 mg of free base/ml), add one equivalent of concentrated sulfuric acid. Stir the reaction mixture at this temperature 10 minutes. After cooling, collect the precipitate by filtration and dry in a vacuum oven at 50° C. overnight.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

For example, Compound I can be administered in a total daily dose of up to 800 mg. Compound I can be administered once daily (QD), or divided into multiple daily doses such as twice daily (BID), and three times daily (TID). Compound I can be administered at a total daily dosage of up to 800 mg, e.g., 200 mg, 300 mg, 400 mg, 600 mg or 800 mg, which can be administered in one daily dose or can be divided into multiple daily doses as described above.

In addition, the administration can be continuous, i.e., every day, or intermittently. The terms “intermittent” or “intermittently” as used herein means stopping and starting at either regular or irregular intervals. For example, intermittent administration of Compound I may mean administration one to six days per week or it may mean administration in cycles (e.g. daily administration for two to eight consecutive weeks, then a rest period with no administration for up to one week) or it may mean administration on alternate days.

Compound I may be administered to the patient at a total daily dosage of between 25-4000 mg/m². In one embodiment, the treatment protocol comprises continuous administration (i.e., every day), once, twice or three times daily at a total daily dose in the range of about 200 mg to about 600 mg.

In another embodiment, the treatment protocol comprises intermittent administration of between three to five days a week, once, twice or three times daily at a total daily dose in the range of about 200 mg to about 600 mg.

In another particular embodiment, Compound I is administered continuously once daily at a dose of 400 mg or twice daily at a dose of 200 mg.

In another particular embodiment, Compound I is administered intermittently three days a week, once daily at a dose of 400 mg or twice daily at a dose of 200 mg.

In another particular embodiment, Compound I is administered intermittently four days a week, once daily at a dose of 400 mg or twice daily at a dose of 200 mg.

In another particular embodiment, Compound I is administered intermittently five days a week, once daily at a dose of 400 mg or twice daily at a dose of 200 mg.

In another particular embodiment, Compound I is administered continuously once daily at a dose of 600 mg, twice daily at a dose of 300 mg, or three times daily at a dose of 200 mg.

In another particular embodiment, Compound I is administered intermittently three days a week, once daily at a dose of 600 mg, twice daily at a dose of 300 mg, or three times daily at a dose of 200 mg.

In another particular embodiment, Compound I is administered intermittently four days a week, once daily at a dose of 600 mg, twice daily at a dose of 300 mg, or three times daily at a dose of 200 mg.

In another particular embodiment, Compound I is administered intermittently five days a week, once daily at a dose of 600 mg, twice daily at a dose of 300 mg, or three times daily at a dose of 200 mg.

In addition, Compound I may be administered according to any of the schedules described above, consecutively for a few weeks, followed by a rest period. For example, Compound I may be administered according to any one of the schedules described above from two to eight weeks, followed by a rest period of one week, or twice daily at a dose of 300 mg for three to five days a week. In another particular embodiment, Compound I is administered three times daily for two consecutive weeks, followed by one week of rest.

Intravenously, the patient would receive Compound I in quantities sufficient to deliver between about 3-1500 mg/m² per day, for example, about 3, 30, 60, 90, 180, 300, 600, 900, 1200 or 1500 mg/m² per day. Such quantities may be administered in a number of suitable ways, e.g. large volumes of low concentrations of Compound I during one extended period of time or several times a day. The quantities can be administered for one or more consecutive days, intermittent days or a combination thereof per week (7 day period).

Alternatively, low volumes of high concentrations of Compound I can be administered during a short period of time, e.g. once a day for one or more days either consecutively, intermittently or a combination thereof per week (7 day period). For example, a dose of 300 mg/m² per day can be administered for 5 consecutive days for a total of 1500 mg/m² per treatment. In another dosing regimen, the number of consecutive days can also be 5, with treatment lasting for 2 or 3 consecutive weeks for a total of 3000 mg/m² and 4500 mg/m² total treatment.

In an embodiment, Compound I can be administered intravenously for a 5-day continuous infusion at 24-64 mg/m²/hr with a cycle duration every 14-21 days or 21-28 days. In another embodiment, Compound I can be administered intravenously for a 5-day continuous infusion at 6-12 mg/m²/hr with a cycle duration every 14-21 days or 21-28 days. In another embodiment, Compound I can be administered intravenously for a 5-day continuous infusion at 8-10 mg/m²/hr with a cycle duration every 14-21 or 21-28 days. In another embodiment, Compound I can be administered intravenously for a 24 hr infusion every 14-21 days at 32-200 mg/m²/hr. In another embodiment, Compound I can be administered intravenously for a 24 hr infusion every 14-21 days at 32-64 mg/m²/hr. In another embodiment, Compound I can be administered intravenously for a 48 hr infusion every 21-28 days at 8-12 mg/M²/hr. In another embodiment, Compound I can be administered intravenously for a 6 hr infusion every 14-21 days at 32-200 mg/m²/hr. In another embodiment, Compound I can be administered intravenously for a 6 hr infusion every 14-21 days at 32-64 mg/m²/hr. In another embodiment, Compound I can be administered intravenously for a 3 hr infusion every 14-21 days at 32-200 mg/m²/hr. In another embodiment, Compound I can be administered intravenously for a 3 hr infusion every 14-21 days at 32-64 mg/m²/hr.

In embodiments, dosage regimens may be combined. In an embodiment, Compound I may be administered at a dosage level or rate for a first specified cycle, such as a five-day infusion every two weeks, over an initial dosage period, such as three months, followed by administration over a second specified cycle, such as a one-day infusion every month, for subsequent maintenance therapy.

Typically, an intravenous formulation may be prepared which contains a concentration of Compound I of between about 1.0 mg/mL to about 10 mg/mL, e.g. 2.0 mg/mL, 3.0 mg/mL, 4.0 mg/mL, 5.0 mg/mL, 6.0 mg/mL, 7.0 mg/mL, 8.0 mg/mL, 9.0 mg/mL and 10 mg/mL and administered in amounts to achieve the doses described above. In one example, a sufficient volume of intravenous formulation can be administered to a patient in a day such that the total dose for the day is between about 300 and about 1500 mg/m².

Any one or more of the specific dosages and dosage schedules for Compound I, is also applicable to any one or more of the anti-cancer agents, anti-proliferative agents, chemotherapeutic agents or Bcr-Abl inhibitors to be used in the combination treatment.

Moreover, the specific dosage and dosage schedule of the anti-cancer agent, anti-proliferative agents, chemotherapeutic agent or Bcr-Abl inhibitor can further vary, and the optimal dose, dosing schedule and route of administration will be determined based upon the specific anti-cancer agent, anti-proliferative agent, chemotherapeutic agent or Bcr-Abl inhibitor that is being used.

Of course, the route of administration of Compound I is independent of the route of administration of the anti-cancer agent, anti-proliferative agent, chemotherapeutic agent or Bcr-Abl inhibitor. In an embodiment, the administration for Compound I is oral administration. In another embodiment, the administration for Compound I is intravenous administration. Thus, in accordance with these embodiments, Compound I is administered orally or intravenously, and the second agent (anti-cancer agent, anti-proliferative agent, chemotherapeutic agent or Bcr-Abl inhibitor) can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form.

In addition, Compound I and anti-cancer agent, anti-proliferative agent, chemotherapeutic agent or Bcr-Abl inhibitor may be administered by the same mode of administration, i.e. both agents administered e.g. orally, by IV. However, it is also within the scope of the present invention to administer Compound I by one mode of administration, e.g. IV, and to administer the anti-cancer agent, anti-proliferative agent, chemotherapeutic agent or Bcr-Abl inhibitor by another mode of administration, e.g. oral or any other ones of the administration modes described hereinabove.

The first treatment procedure, administration of Compound I, can take place 1) prior to the second treatment procedure, i.e., the anti-cancer agent, anti-proliferative agent, chemotherapeutic agent or Bcr-Abl inhibitor; 2) after the treatment with the anti-cancer agent, anti-proliferative agent, chemotherapeutic agent or Bcr-Abl inhibitor; 3) at the same time as the treatment with the anti-cancer agent, anti-proliferative agent, chemotherapeutic agent or Bcr-Abl inhibitor; or 4) a combination thereof. For example, a total treatment period can be decided for Compound I. The anti-cancer agent, anti-proliferative agent, chemotherapeutic agent or Bcr-Abl inhibitor can be administered prior to onset of treatment with Compound I or following treatment with Compound I. In addition, anti-cancer agent, anti-proliferative agent, chemotherapeutic agent or Bcr-Abl inhibitor treatment can be administered during the period of Compound I administration but does not need to occur over the entire Compound I treatment period.

Compound I can be administered in accordance with any dose and dosing schedule that, together with the effect of the anti-cancer agent, anti-proliferative agent, chemotherapeutic agent or Bcr-Abl inhibitor, achieves a dose effective to treat cancer.

For a specific example of the administration of compound I, see Example 7.

Depending upon the particular condition, or disease, to be treated or prevented, additional therapeutic agents, which are normally administered to treat or prevent that condition, may also be present in the compositions of this invention. In some embodiments, additional therapeutic agents may be co-administered or administered sequentially with Compound I to treat a patient in need thereof. Some embodiments comprise administering to a patient in need thereof a first amount of Compound I, in a first treatment procedure, and a second amount of an additional therapeutic agent in a second treatment procedure. In some embodiments, said additional therapeutic agent is selected from an anti-cancer agent, an anti-proliferative agent, a chemotherapeutic agent or an inhibitor of Bcr-Abl. The first and second treatments together comprise a therapeutically effective amount.

In some embodiments, administration of Compound I is oral administration. In other embodiments, administration of Compound I is intravenous administration. As used herein, additional therapeutic agents that are normally administered to treat or prevent a particular disease, or condition, are known as “appropriate for the disease, or condition, being treated”.

For example, chemotherapeutic agents or other anti-proliferative agents may be combined with Compound I to treat proliferative diseases and cancer. Examples of known chemotherapeutic agents include, but are not limited to, Gleevec™, adriamycin, dexamethasone, vincristine, cyclophosphamide, fluorouracil, topotecan, taxol, interferons, and platinum derivatives.

Other therapies or anticancer agents that may be used in combination with the inventive anticancer agents of the present invention include surgery, radiotherapy (in but a few examples, gamma-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), Gleevec™, adriamycin, dexamethasone, and cyclophosphamide. For a more comprehensive discussion of updated cancer therapies see, http://www.nci.nih.gov/, a list of the FDA approved oncology drugs at http://www.fda.gov/cder/cancer/druglistframe.htm, and The Merck Manual, Seventeenth Ed. 1999, the entire contents of which are hereby incorporated by reference.

In one embodiment, inhibitors of Bcr-Abl may be combined with Gleevec™ to treat proliferative diseases and cancer.

In another embodiment, inhibitors of Bcr-Abl may be combined with Gleevec™ to treat proliferative diseases and cancer, wherein the inhibitor of Bcr-Abl is selected from: SKI-606, BMS354825, AZDO530, AP23464, CGP76030 and AMN107.

In another embodiment, inhibitors of wild type Abl kinase may be combined with Gleevec™ to treat proliferative diseases and cancer.

In another embodiment, inhibitors of mutant Abl kinase may be combined with Gleevec™ to treat proliferative diseases and cancer.

In another embodiment, inhibitors of T315I Abl kinase may be combined with inhibitors of Bcr-Abl which are selected from: SKI-606, BMS354825, AZDO530, AP23464, CGP76030, AMN107 and Gleevec™ to treat proliferative diseases and cancer.

In another embodiment, inhibitors of T315I Abl kinase may be combined with inhibitors of Bcr-Abl which are selected from: SKI-606, BMS354825, AZDO530, AP23464, CGP76030, AMN107 and Gleevec™ to treat CML and ALL.

In another embodiment, inhibitors of T315I Abl kinase may be combined with Gleevec™ to treat proliferative diseases and cancer.

In another embodiment, inhibitors of T315I Abl kinase may be combined with inhibitors of Bcr-Abl which are selected from: SKI-606, BMS354825, AZD0530, AP23464, CGP76030, AMN107 and Gleevec™ to treat proliferative diseases and cancer, wherein the T315I inhibitor is Compound I.

In another embodiment, inhibitors of T315I Abl kinase may be combined with inhibitors of Bcr-Abl which are selected from: SKI-606, BMS354825, AZDO530, AP23464, CGP76030, AMN107 and Gleevec™ to treat CML and ALL, wherein the T315I inhibitor is Compound I.

In another embodiment, inhibitors of T315I Abl kinase may be combined with Gleevec™ to treat proliferative diseases and cancer, wherein the T315I inhibitor is Compound I.

In another embodiment, inhibitors of T315I Abl kinase may be combined with Gleevec™ to treat CML and ALL, wherein the T315I inhibitor is Compound I.

In another embodiment, inhibitors of T315I Abl kinase may be combined with Gleevec™ to treat CML, wherein the T315I inhibitor is Compound I.

In another embodiment, Compound I may be used in combination with Dasatinib (BMS354825) for the treatment of leukemia.

In another embodiment, Compound I may be used in combination with Dasatinib (BMS354825) for the treatment of CML.

In another embodiment, Compound I may be used in combination with Dasatinib (BMS354825) for the treatment of T315I CML.

In another embodiment, Compound I may be used in combination with Dasatinib (BMS354825) for the treatment of ALL.

In another embodiment, Compound I may be used in combination with Dasatinib (BMS354825) for the treatment of Philadelphia+ALL.

In another embodiment, Compound I may be used in combination with Nilotinib (AMN107) for the treatment of leukemia.

In another embodiment, Compound I may be used in combination with Nilotinib (AMN107) for the treatment of CML.

In another embodiment, Compound I may be used in combination with Nilotinib (AMN107) for the treatment of T315I CML.

In another embodiment, Compound I may be used in combination with Nilotinib (AMN107) for the treatment of ALL.

In another embodiment, Compound I may be used in combination with Nilotinib (AMN107) for the treatment of Philadelphia+ALL.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

If Compound I is used in combination with an additional agent, the additional agent may be used in the same (i.e., a single) dosage form or in separate dosage forms.

In order that the invention described herein may be more fully understood, the following examples are set forth. It should be understood that these examples are for illustrative purposes only and are not to be construed as limiting this invention in any manner.

### EXAMPLES

Examples 1-4 refer to the compounds of the General Scheme above.

### Example 1

### 4,6-Dichloropyrimidine-2-methylsulfone (A)

Prepared by methods substantially similar to those set forth in Koppell et al, JOC, 26, 1961, 792, in the following manner. To a stirred solution of 4,6-dichloro-2-(methylthio)pyrimidine (50 g, 0.26 mol) in dichloromethane (1 L) at 0° C. was added meta-chloroperoxybenzoic acid (143.6 g, 0.64 mol) over a period of 20 minutes. The solution was allowed to warm to room temperature and was stirred for 4 hours. The mixture was diluted with dichloromethane (1.5 L) and then treated sequentially with 50% Na₂S₂O₃/NaHCO₃ solution (2×200 ml), sat. NaHCO₃ solution (4×300 ml), and brine (200 ml) then dried (MgSO₄). The solvent was removed in vacuo to afford an off-white solid, which was redissolved in EtOAc (1 L) and treated sequentially with sat. NaHCO₃ solution (3×300 ml), and brine (100 ml) then dried (MgSO₄). The solvent was removed in vacuo to afford the title compound (A) as a white solid (55.6 g, 96% yield). ¹H NMR CDCl₃ δ 3.40 (3H, s, CH3), 7.75 (1H, s. ArH).

### Example 2

### Cyclopropane carboxylic acid [4-(4,6-dichloro-pyrimidin-2-ylsulphanyl)-phenyl]-amide (C)

A suspension of compound A (10 g, 44.04 mmol) and cyclopropane carboxylic acid (4-mercapto-phenyl)-amide (B, 8.51 g, 44.04 mmol) in t-butanol (300 ml) was degassed by evacuation, then flushing with nitrogen. The mixture was stirred at 90° C. under nitrogen atmosphere for 1 hour then the solvent was removed in vacuo. The residue was dissolved in ethyl acetate (600 ml) and washed with an aqueous solution of potassium carbonate and sodium chloride. The organic extract was dried over magnesium sulphate, concentrated to a low volume and allowed to crystallize. The product C was collected as colourless crystals, (11.15 g, 74%). ¹H-NMR DMSO-d⁶, δ0.82-0.89 (4H, m), 1.80-1.88 (1H, m), 7.55 (2H, d), 7.70-7.76 (3H, m), 10.49 (1H, s); M+H, 340.

### Example 3

### Cyclopropane carboxylic acid{4-[4-chloro-6-(5-methyl-2H-pyrazol-3-ylamino)-pyrimidin-2-ylsulphanyl]-phenyl} amide (D)

A mixture of compound C (1.0 g, 2.94 mmol) and 3-amino-5-methylpyrazole (314 mg, 3.23 mmol) in dimethylformamide (6 ml) was treated with diisopropylethylamine (0.614 ml, 3.53 mmol) and sodium iodide (530 mg, 3.53 mmol). The mixture was stirred under nitrogen at 85° for 4 hours, cooled to room temperature and diluted with ethyl acetate. The solution was washed with water (×4), dried over magnesium sulphate and concentrated to 5 ml to afford, upon crystallization and harvesting of colourless crystals, the title compound D (920 mg, 78%). ¹H-NMR DMSO-d⁶, δ 0.80-0.87 (4H, m), 1.77-1.85 (1H, m), 1.92 (1H, s), 5.24 (1H, br s), 6.47 (1H, br s), 7.55 (2H, d), 7.70-7.80 (2H, m), 10.24 (1H, s), 10.47 (1H, s), 11.92 (1H, s).

### Example 4

### Cyclopropane carboxylic acid {4-[4-(4-methyl-piperazin-1-yl)-6-(5-methyl-2H-pyrazol-3-ylamino)-pyrimidin-2-ylsulphanyl]-phenyl}-amide (I)

Compound D (2.373 g, 5.92 mmol) was treated with N-methylpiperazine (10 ml) and the mixture stirred at 110° for 2 hours. The excess N-methylpiperazine was removed in vacuo then the residue was dissolved in ethyl acetate, washed with aqueous sodium bicarbonate solution, dried over magnesium sulphate, and concentrated. The residue was crystallised from methanol to give colourless crystals of desired product I (1.82 g, 66%), ¹H-NMR DMSO-d⁶, δ 0.81 (4H, d), 1.79 (1H, m), 2.01 (3H, s), 2.18 (3H, s), 2.30 (4H, m), 3.35 (masked signal), 5.42 (11H, s), 6.02 (1H, br s), 7.47 (2H, d), 7.69 (2H, d), 9.22 (1H, s), 10.39 (1H, s), 11.69 (1H, s).

### Example 5

### Abl Kinase Activity Inhibition Assay and Determination of the Inhibition Constant Ki

Compounds were screened for their ability to inhibit N-terminally truncated (Δ 27) Abl kinase activity using a standard coupled enzyme system (Fox et al., Protein Sci. 7, pp. 2249 (1998)). Reactions were carried out in a solution containing 100 mM HEPES (pH 7.5), 10 mM MgCl₂, 25 mM NaCl, 300 μM NADH, 1 mM DTT and 3% DMSO. Final substrate concentrations in the assay were 110 μM ATP (Sigma Chemicals, St Louis, Mo.) and 70 μM peptide (EAIYAAPFAKKK, American Peptide, Sunnyvale, Calif.). Reactions were carried out at 30° C. and 21 nM Abl kinase. Final concentrations of the components of the coupled enzyme system were 2.5 mM phosphoenolpyruvate, 200 μM NADH, 60 μg/ml pyruvate kinase and 20 μg/ml lactate dehydrogenase.

An assay stock buffer solution was prepared containing all of the reagents listed above with the exception of ATP and the test compound of interest. The assay stock buffer solution (60 μl) was incubated in a 96 well plate with 2 μl of the test compound of interest at final concentrations typically spanning 0.002 μM to 30 μM at 30° C. for 10 min. Typically, a 12 point titration was prepared by serial dilutions (from 1 mM compound stocks) with DMSO of the test compounds in daughter plates. The reaction was initiated by the addition of 5 μl of ATP (final concentration 110 μM). Rates of reaction were obtained using a Molecular Devices Spectramax plate reader (Sunnyvale, Calif.) over 10 min at 30° C. The Ki values were determined from the residual rate data as a function of inhibitor concentration using nonlinear regression (Prism 3.0, Graphpad Software, San Diego, Calif.).

### Example 6

### Mutant Abl Kinase (T315I) Activity Inhibition Assay and Determination of the Inhibition Constant IC50

Compounds were screened for their ability to inhibit the T315I mutant form of human Abl at Upstate Cell Signaling Solutions (Dundee, UK). In a final reaction volume of 25 μl, the T315I mutant of human Abl (5-10 mU) was incubated with 8 mM MOPS pH 7.0, 0.2 mM EDTA, 50 μM EAIYAAPFAKKK, 10 mM Mg Acetate, [γ-³³P-ATP] (specific activity approx. 500 cpm/pmol, 10 mM final assay concentration) and the test compound of interest at final concentrations over the range 0-4 μnM. The reaction was initiated by the addition of the MgATP mix. After incubation for 40 minutes at room temperature, the reaction was stopped by the addition of 5 μl of a 3% phosphoric acid solution. 10 μl of the reaction was then spotted onto a P30 filtermat and washed three times for 5 minutes in 75 mM phosphoric acid and once in methanol prior to drying and scintillation counting. Inhibition IC50 values were determined from non-linear regression analysis of the residual enzyme activities as a function of inhibitor concentration (Prism 3.0, Graphpad Software, San Diego, Calif.).

### Example 7

A phase I/II study of MK-0457 (also known as Compound I or VX-680) was initiated in June 2005. Eligible patients initially included those with refractory AML or ALL. Patients were treated with a 5-day CIV infusion at 2 to 3 week intervals. The dose of MK-0457 was escalated in successive cohorts of three patients per dose level. If none of the first 3 patients at a dose level experienced first cycle dose-limiting toxicity (DLT), then 3 new patients could be entered at the next higher dose level. If 1 of 3 patients experienced first cycle DLT, up to 3 more patients would be started at that same dose level (total N=6). If 2 or more experienced first cycle DLT, no further patients were started at that dose. The MTD (highest dose level in which <2 patients of 6 developed first cycle DLT) was not reached. Each new dose levels could begin accrual only if all patients at the current dose level had been observed for a minimum of 14 days from the last day of infusion. The recommended Phase II dose (RP2D) was considered to be the MTD unless significant clinical activity was seen below the MTD.

PCR-based DNA sequencing of BCR-ABL codons 221 to 500 of the kinase domain was used to detect mutations. The protocol was approved by the MD Anderson Cancer Center (MDACC) Institutional Review Board and all patients provided written informed consent.

Dose levels of 10, 14, 18, 20 and 28 mg/m²/hour have been investigated. No grade 3 or 4 potentially MK-0457-attributable adverse events have been observed in 22 evaluable patients. The first patient with a T315I BCR-ABL mutation treated on study was a 53-year-old male diagnosed with Ph chromosome-positive CML in November 2001 with a presenting WBC of 400×10⁹/L. Baseline karyotype also showed a derivative chromosome 22. The patient commenced therapy with imatinib 400 mg per day and achieved a complete hematologic response (CHR) of 15 months duration. In May 2003, he lost CHR and commenced imatinib at 600 mg per day. He had never achieved a major cytogenetic remission. In June 2003, the WBC was 430×10⁹/L despite additional Hydroxyurea, and the patient was referred to MDACC. Karyotype showed multiple Ph chromosomes in a minority of cells. The patient declined investigational therapy or stem cell transplantation and was treated with imatinib, Hydroxyurea, and pegylated alpha interferon until March 2005. The WBC was not controlled and no degree of cytogenetic response was achieved on this regimen. The patient returned to MDACC with accelerated phase CML and commenced therapy with nilotinib 600 mg BID in April 2005. The patient had a transient decrease in WBC, then required increasing doses of Hydroxyurea, and had clearly failed to respond to nilotinib when this was discontinued in July 2005. At this time, the patient was first reported to have the T315I bcr-abl mutation. The patient was then treated on protocol with KOS-953 (17-allylamino-17-demethoxy-geldanamycin), a HSP-90 inhibitor. (Georgalis G V, Younes A: Heat-shock protein 90 inhibitors in cancer therapy: 17AAG and beyond. Future Oncol 2005; 1: 273-81.) The patient received four courses of therapy of KOS-953 with imatinib but required increasing doses of Hydroxyurea and stopped protocol therapy in October 2005. He subsequently commenced therapy with MK-0457 at a dose of 12 mg/m²/hour CIV daily for five days in November 2005. By Day 11 of cycle 1, the patient was pancytopenic with WBC of 0.4×10⁹/L, Hb of 7.6 g/dl and platelet count of 31×10⁹/L. These counts rapidly recovered and 2 weeks later on day 1 of cycle 2 of therapy, the WBC was 77×10⁹/L, Hb was 12 g/dl, and platelets 698×10⁹/L. In the initial four cycles, this pattern was repeated with a decrease in counts with each therapy and a subsequent rise with a steady increase in the platelet count to >1000×10⁹/L by end of cycle 4 at which time anagrelide 0.5 mg BID was added. Cycle 6 of therapy began in February 2006 at a dose of 16 mg/m²/hour CIV daily for 5 days. Cycle 10 commenced at a dose of 20 mg/m²/hour CIV daily for 5 days in April 2006 by which time the patient had a normal platelet count in the absence of anagrelide therapy. At this time the patient was returned to chronic phase with a normal CBC in the absence on Hydroxyurea or anagrelide therapy which has not been possible in the prior three years. The patient continues on MK-0457 therapy at three to four week intervals. The T315I clone continues to be predominant in the bone marrow which continues to be predominantly Ph chromosome positive.

The second patient with a T315I bcr-abl mutation treated on study was a 33 year old female who was diagnosed with Ph-positive CML in 1997. She initially received therapy with Hydroxyurea and alpha interferon alone for 6 months. In 1998, she commenced therapy with imatinib which she received at doses of 400 mg to 800 mg daily until August 2005, at which time she clearly had failed to achieve a durable CHR and was treated on protocol with dasatinib. After a transient response, she was taken off study in October 2005 secondary to lack of response. She was then referred to MDACC with refractory accelerated phase disease for evaluation for therapy on a nilotinib protocol. At this time, the patient was first reported to have the T315I BCR-ABL mutation. The patient commenced therapy with MK-0457 at a dose of 16 mg/m²/hour CIV daily for five days in January 2006. As in the first patient, an initial decrease in blood counts was followed by a subsequent rise with a steady increase in the platelet count to >1000×10⁹/L by end of cycle 2 at which time anagrelide 0.5 mg BID was added. Repeat PCR-based DNA sequencing of BCR-ABL no longer detected the presence of the T315I mutation after cycle 1 of therapy. After cycle 2 of therapy the patient could no longer stay on protocol for social reasons and wished to attempt cytotoxic therapy in her local hospital.

A third patient with the T315I BCR-ABL mutation was a 63 year old male diagnosed with Ph chromosome-positive ALL in December 2003. He achieved CHR to standard induction therapy and received both systemic and intrathecal consolidation therapy. No cytogenetic response was achieved and in September 2005, overt relapse was evident. He then began protocol therapy with dasatinib 70 mg BID. He achieved CHR and a diploid karyotype by November 2005. In January 2006 the hematologic and cytogenetic responses were lost and the dasatinib dose increased to 90 mg BID. At this dose the patient had recurrent lower GI bleeding and dasatinib was discontinued in February 2006. The patient was then referred to MDACC and was first reported to have the T315I BCR-ABL mutation. The patient commenced therapy with MK-0457 at a dose of 20 mg/m²/hour CIV daily for five days in March 2006. At time of study entry the patient had fungal pneumonia and a WBC of 15×10⁹/L with 81% blasts. Following 2 cycles of therapy the patient had a WBC of 1.6×10⁹/L with 88% neutrophils, no blasts. The fungal pneumonia began to respond to systemic anti-fungal therapy associated with neutrophil recovery and further MK-0457 therapy was planned.

While a number of embodiments of this invention have been described, it is apparent that the basic examples may be altered to provide other embodiments, which utilize the compounds and methods of this invention. Therefore, it will be appreciated that the scope of this invention is to be defined by the appended claims rather than by the specific embodiments, which have been represented by way of example.

We claim:

1. A method for inhibiting Abl kinase comprising contacting Compound I and the Abl kinase.

2. The method according to claim 1, wherein the Abl kinase is wild-type kinase.

3. The method according to claim 1, wherein the Abl kinase is a mutant form of the Abl kinase.

4. The method according to claim 3, wherein the mutant form of the Abl kinase is a T315I mutant.

5. A method for treating CML in a patient comprising administering to the patient a therapeutically effective amount of Compound I, or a pharmaceutically acceptable salt thereof.

6. A method for treating ALL in a patient comprising administering to the patient a therapeutically effective amount of Compound I, or a pharmaceutically acceptable salt thereof.

7. A method for inhibiting an Abl kinase in a patient in need thereof comprising administering a therapeutically effective amount of Compound I to the patient.

8. The method according to claim 7, wherein the Abl kinase is a mutant Abl kinase.

9. The method according to claim 8, wherein the mutant Abl kinase is a T315I mutant.

10. A therapeutic method comprising determining whether a T315I Abl mutation is present in a patient, and, if the T315I Abl mutation is present, administering Compound I to the patient.
